(19) United States
(12) Patent Application Publication
(10) Pub. No.: US 2007/0264360 A1
(43) Pub. Date: Nov. 15, 2007

(54) Gall bladder and liver cleansing suppository

(76) Inventor: Spencer B. Feldman, Kula, HI (US)

Correspondence Address: SPENCER FELDMAN, 18 Stuyuesant Oval, New York, NY, 10009, US

(21) Appl. No.: 11/731,506

(22) Filed: Mar. 30, 2007

Related U.S. Application Data
(60) Provisional application No. 60/799,854, filed on May 12, 2006.

(51) Int. Cl. A61K 31/13; A61K 31/195; A61K 31/661; A61K 36/00; A61K 36/233; A61P 1/16

(52) U.S. Cl. 424/725; 424/773; 514/114; 514/561; 514/665

(57) ABSTRACT

A composition containing any of the following alone or in combination: Glycine, taurine, phosphatidyl choline, Bupleurum and or Chanca Piedra in a suppository.

### PARENT CASE TEXT

This application claims priority from provisional patent application No. 60/799,854

### DESCRIPTION

### Field of the Invention

The present invention relates to a composition containing singly or in combination, the amino acids glycine, taurine, the phospholipid phosphatidyl choline, and the herbs Bupleurum and Chanca Piedra administered in suppository form.

### BACKGROUND OF THE INVENTION

Gallstones account for approximately 600,000 hospitalizations and 500,000 operations per year in the USA. A non-surgical treatment of gallstones is offered here.

The predominant bile salts in the body are glycocholate and taurocholate. They are formed from the amino acids glycine and taurine. These along with phosphatidyl choline are the main components in the maintaining bile solubility. Insufficient levels of these compounds precipitate the formation of biliary sludge and gallstones, while sufficient level of these nutrients can prevent and even reverse these conditions. The addition of the herbs Bulpeurum and Chanca Piedra also helps stimulate the production of bile and removal of gallstones.

When administered as a suppository, these ingredients are delivered more directly to the liver via the hemorrhoidal complex and portal vein than when taken orally. In addition, upset stomach is also avoided.

### SUMMARY OF THE INVENTION

The present invention provides a non-surgical treatment of gallstones by dissolving the gallstones in-vivo using the amino acids glycine, taurine, the phospholipid phosphatidyl choline and the herbs Chanca Piedra and Bupleurum.

### DETAILED DESCRIPTION OF THE INVENTION

According to this invention, a suppository is formed of the amino acids glycine, taurine, the pospholipid phosphatidyl choline and the herbs Chanca Piedra and Bupleurum and a matrix. The suppository is molded in a common shape from a waxy material in which active ingredients have been dissolved or suspended. The base material may comprise cocoa butter, glycerin, glyceryl, monopalmitate, glyceryl monostearate, hydrogenated coconut oil fatty acids and hydrogenated palm kernel oil fatty acids or polyethylene glycol. The choice of base material is a matter of ordinary skill.

### Preferred Method

120 mg of glycine, 120 mg of taurine, 120 mg of phosphatidyl choline, 120 mg of Bupleurum, 120 mg Chanca Piedra are mixed together and put in a cocoa butter base.

I claim:

1. A method of administering the amino acid glycine to a patient, comprising the steps of: (a) forming a suppository consisting essentially of containing the amino acid glycine and (b) administering said suppository to the patient.

2. A method of administering the amino acid taurine to a patient, comprising the steps of: (a) forming a suppository consisting essentially of containing the amino acid taurine and (b) administering said suppository to the patient.

3. A method of administering the phospholipid phosphatidyl choline to a patient, comprising the steps of: (a) forming a suppository consisting essentially of containing the phospholipid phosphatidyl choline and (b) administering said suppository to the patient.

4. A method of administering the herb Chanca Piedra to a patient, comprising the steps of: (a) forming a suppository consisting essentially of containing Chanca Piedra and (b) administering said suppository to the patient.

5. A method of administering the herb Bupleurum to a patient, comprising the steps of: (a) forming a suppository consisting essentially of containing Bupluerum and (b) administering said suppository to the patient.
